(19) United States
(12) Patent Application Publication
(10) Pub. No.: US 2019/0183671 A1
(43) Pub. Date: Jun. 20, 2019

(54) HOT/COLD TEMPERATURE TREATMENT APPARATUS

(71) Applicant: Hygenic Intangible Property Holding Co., Akron, OH (US)

(72) Inventor: Carol Baltazar, Glenwood, MD (US)

(21) Appl. No.: 15/844,977

(22) Filed: Dec. 18, 2017

(51) Int. Cl. A61F 7/02; C09K 5/10

(52) U.S. Cl. CPC A61F 7/02; A61F 2007/0003; C09K 5/10

(57) ABSTRACT

Packs for providing heat or cold to a body part or parts. In some embodiments, packs comprising an outer flexible shell or skin housing or carrying a plurality of beads mixed in combination with a liquid or gel. In some embodiments, packs, which can be (or are) configured into various shapes, which contain heads in combination with a liquid or gel solution, which can be heated or cooled for application, thereafter, to a body part for providing hot or cold therapy to the body part.

### RELATED APPLICATION DATA

This application claims priority to U.S. Provisional Patent Application No. 61/184,788, filed Jun. 6, 2009, similarly titled, the entirety of which is hereby incorporated by reference.

### FIELD OF INVENTION

Packs for providing heat or cold to a body part or parts. In some embodiments, packs comprising an outer flexible shell or skin housing or carrying a plurality of beads mixed in combination with a liquid or gel.

### BACKGROUND OF THE INVENTION

The use of hot or cold temperature treatments for therapeutic or medical use is well known in the art. For example, it is well known that cold therapy is helpful for reducing swelling and inflammation (e.g., by reducing blood flow to an injured body area) and that heat therapy is useful for relieving pain which may occur due to bodily injury or trauma (for example, a sprain or other sport injury) or, for example, for treating localized arthritis pain. Although water bottles and electric heating pads for providing heat (for example) to body parts have been available for decades as have been ice packs and gel packs for providing cold therapy, there exists a long felt need in the art for further improvements in hot and cold therapies.

In view of the desires for improvements in the art, it is a purpose of the herein described invention to address one or more of such desires as well as, or in the alternative, other needs which will become more apparent to the skilled artisan once given the present disclosure.

### SUMMARY OF THE INVENTION

Generally speaking, this invention relates to packs, which can be configured into various shapes, which contain beads in combination with a liquid or gel solution, which can be heated or cooled for application, thereafter, to a body part for providing hot or cold therapy to the body part. In certain embodiments, the packs are configured to conform to particular body parts such as a facial area (e.g., over the eyes), ankles, knees, shoulders, elbows, the head, or the like (by providing different shapes, sizes, and/or degrees of flexibility such that the packs will conform to a particular body part or parts).

In some embodiments, the packs contain cylindrical beads, which are optionally, but preferably, deformable. In certain embodiments, such beads are contained or carried in a sealed flexible pack or shell such as comprised of a durable plastic or PVC type material (or any other material of suitable type). In certain embodiments, such as described above or in other configurations or combinations, cylindrical beads are used as a filler material in a sealed, flexible pack or shell in combination with a select amount of liquid or gel material. In certain embodiments, the majority of the filler material is comprised of such beads with a liquid or gel added in minor (or other) amounts to aid in temperature retention, to allow for or provide a “fluid” movement or suspension of the beads, and/or for providing cushioning and/or ergonomic and/or other comfort to the user.

In certain embodiments, the beads are hollow, but, in preferred embodiments, the beads are substantially or completely solid in configuration. In certain embodiments, the beads are comprised of a flexible and/or deformable material. In preferred, but optional, embodiments, the beads are comprised of a gel-type material (but may be comprised of other soft-type materials). In one example, the beads are comprised of glycerin (and may contain some binders such as sorbitol). In such or other examples, the beads are contained in a pack formed or comprised of flexible, clear PVC material. In such or other examples, the beads fill the majority of the enclosed pack (e.g., 60-95%) with the remaining space being filled with water or gel (but with some room left within the pack, so that the pack is flexible and/or bendable, and/or deformable and the like). In certain embodiments, the liquid (e.g., water) or gel is used as a suspension. In certain embodiments using water, a bicarbonate buffer solution is added or employed.

In preferred embodiments, the packs described herein are so configured such as to be conformable (substantially or entirely) to the skin and/or various body curves and angles. In still further preferred embodiments, the beads, due to their softness or deformability, aid in the conformable nature of the packs. In still other preferred embodiments, the packs may be so configured, using a particular combination of beads with liquid (e.g., water) or gel (and/or with the beads being comprised of a particular composition, such as gelatin) such that the approximate time of heat or cold dissipation is approximately equal to a time equal to (or at least approximating) a doctor recommended temperature therapy. For example, the packs can be specifically configured so that when heated or cooled, the packs return to a temperature approximating either a room or body temperature in approximately 20 minutes (or, for example, between 17 and 30 minutes or some other desirable time). Configured as such, actual timing by a user or patient of temperature treatments is not needed because the hot or cold properties of the packs will dissipate at or by (or near) the conventional and/or doctor recommended time automatically (at which point, the treatment can be switched to the opposite temperature treatment, or repeated, or simply discontinued, for example). That is, the user or patient will receive the correct time application of the heat or cold temperature treatment but without the need to specifically time the treatment (which allows for more relaxation or for sleeping during treatment, for example).

In at least one embodiment, the packs are comprised of outer flexible shells containing beads filling approximately 80-99 percent of the interior pack area in combination with liquid or gel filling approximately 1-20% of the pack.

In at least one embodiment, there is provided: a hot and cold temperature treatment pack comprising: a sealed pack shell comprised of one or more flexible walls configured so as to provide an interior compartment for carrying a heatable and chillable payload; a plurality of deformable beads carried in the interior compartment; a liquid carried in the interior compartment; wherein the interior compartment is filled with a particular quantity of the deformable beads and a particular quantity of the liquid, and the ratio of deformable beads to the liquid within the interior compartment is selected such that: a) when the treatment pack is heated, the treatment pack remains at a therapeutic, heated temperature for a therapeutically effective time; and b) when the treatment pack is chilled, the treatment pack remains at a therapeutic, cooled temperature for a therapeutically effective time.

In another embodiment, there is provided: a hot and cold temperature treatment pack comprising: a sealed pack shell comprised of one or more flexible walls configured so as to provide an interior compartment for carrying a heatable and chillable payload; a plurality of deformable beads carried in the interior compartment; a liquid carried in the interior compartment; wherein the interior compartment is filled with a particular quantity of the deformable beads and a particular quantity of the liquid, and the ratio of deformable beads to the liquid within the interior compartment is selected such that: a) when the treatment pack is heated, the treatment pack remains at a therapeutic, heated temperature for a therapeutically effective time; and b) when the treatment pack is chilled, the treatment pack remains at a therapeutic, cooled temperature for a therapeutically effective time; and wherein the interior compartment is filled 80-99 percent with the deformable beads and 1-20% with the liquid.

Example uses of the herein described packs are for treating: sprains, muscle aches, post-surgical pain, workout soreness, swelling, puffiness, bruising, arthritis pain, migraines, headaches, toothaches, or they may be used for or with facials and/or other spa treatments.

Certain examples of the invention are now described below with respect to certain non-limiting embodiments thereof as illustrated in the following drawings wherein:

### BRIEF DESCRIPTION OF THE DRAWINGS

FIG. 1 illustrates one embodiment of a temperature treatment pack according to the subject invention.

FIG. 2 illustrates an alternative embodiment of a temperature treatment pack according to the subject invention.

FIG. 3 illustrates a further alternative embodiment of a temperature treatment pack according to the subject invention.

FIG. 4 illustrates yet an additional embodiment of a temperature treatment pack according to the subject invention.

FIG. 5 illustrates a three-dimensional, cross-sectional view of an embodiment of a solid bead used in conjunction with certain embodiments of temperature treatment packs according to the subject invention.

FIG. 6 illustrates a three-dimensional, cross-sectional view of an embodiment of a hollow bead used in conjunction with certain embodiments of temperature treatment packs according to the subject invention.

### DETAILED DESCRIPTION OF CERTAIN EMBODIMENTS

For a more complete understanding of the present invention, reference is now made to the following description of various illustrative and non-limiting embodiments thereof, taken in conjunction with the accompanying drawings in which like reference numbers indicate like features.

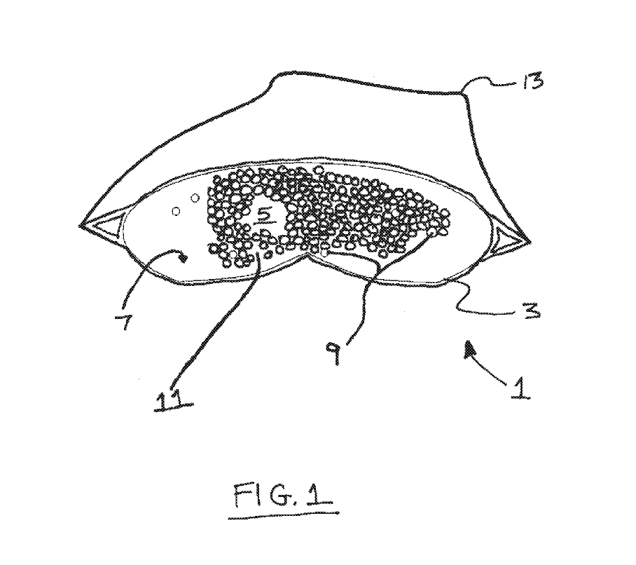

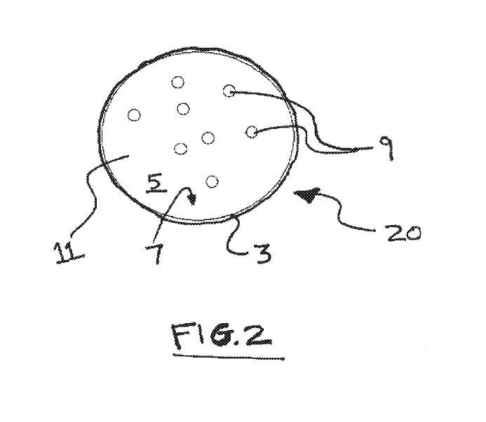

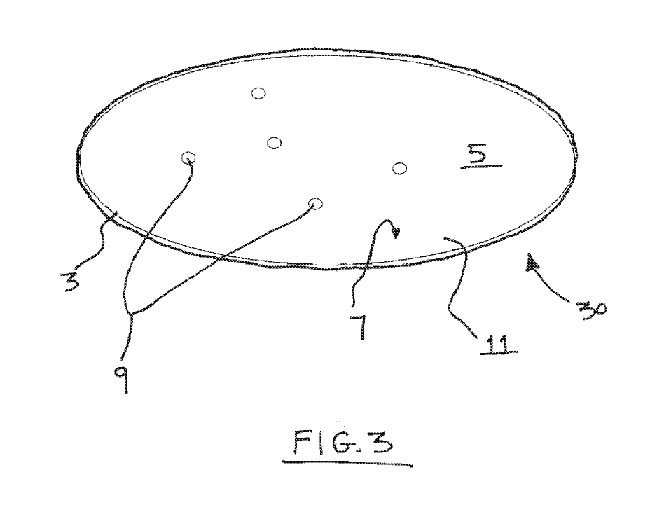

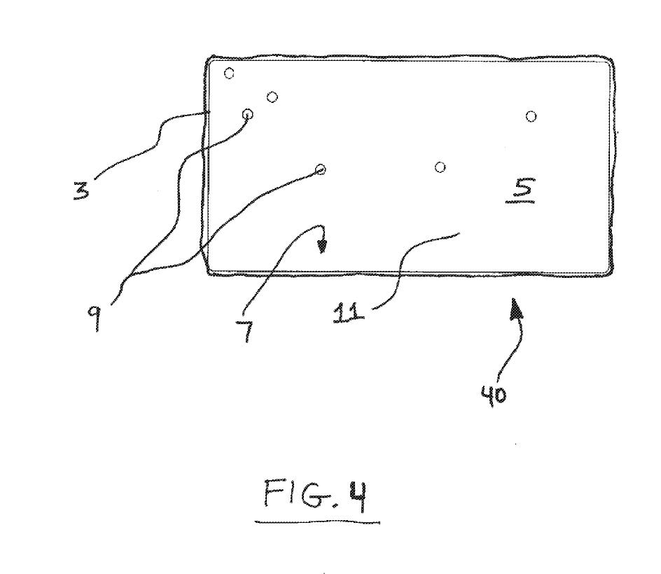

Referring initially to FIGS. 1-4, various non-limiting embodiments of temperature treatment packs illustrative of one or more features of the subject invention are shown therein. In this regard, in FIG. 1, for example, an exemplar temperature treatment pack 1 is illustrated, configured in the shape of an eye mask (example alternative configurations are designated as 20, 30, and 40 in FIGS. 2, 3, and 4, respectively, with all other parts or components being identically labelled), comprised of one or more flexible walls 5 formed into a flexible shell 3. Flexible shell 3, in turn, is configured to have an interior compartment 7 for carrying a heatable and chillable payload. In particular, the payload provided by the subject invention is comprised of a plurality of deformable beads 9 carried in interior compartment 7 mixed with or suspended in a liquid 11, also carried and/or contained in the interior compartment. Although, in the subject figures, some representative quantity of beads 9 are illustrated within the packs, it should be noted that the drawings are representative only and are not intended to illustrate a full or even desirable pack bead load.

Beads 9 are preferably comprised of glycerin and may—but are not required to contain one or more binders such as sorbitol. Other materials for forming such beads may, of course, be employed (e.g., preferably gel-type materials but other soft materials with heat and cold retention properties can be utilized). Shell 3 and its walls 5 are preferably formed from polyvinyl chloride (PVC) but other flexible, durable materials to form such walls and/or shell can be used.

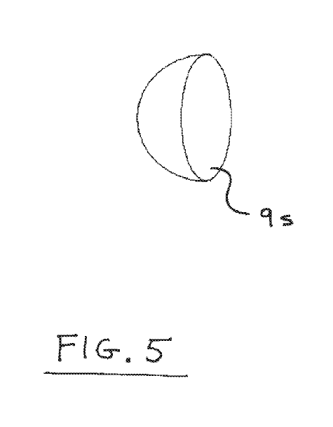

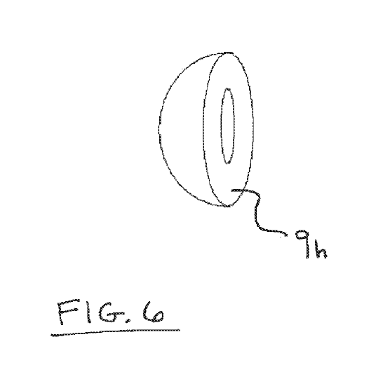

Beads 9 may be solid in configuration such as the bead depicted as 9s in FIG. 5 or, in the alternative, may be hollow such as bead 9h depicted in FIG. 6. Moreover, mixtures of hollow and solid beads can be employed. Notably, tailoring the ratio of solid to hollow beads may done to obtain different values (or times) of heat or cold retention by the temperature treatment packs described herein. Similarly, the ratio of beads 9 to liquid 11 can be tailored or varied to purposefully select temperature retention times so that the temperature treatment packs cool (from a heated temperature) or warm (from a chilled temperature) in or within a desirable time frame such as corresponding to therapeutically effective time frames.

In preferred embodiments of the apparatus described herein, the packs are particularly configured such as to be conformable (substantially or entirely) to the skin and/or various body curves and angles. In further preferred embodiments, beads 9, due to their softness or deformability, aid in the conformable nature of the packs.

In still other preferred embodiments, the packs are purposely configured, using a particular combination or ratio of beads to liquid (e.g., water) or gel (and/or with the beads being comprised of a particular composition, such as glycerin) such that the approximate time of heat or cold dissipation is approximately equal to a time equal to (or at least approximating) a doctor recommended temperature therapy. For example, certain particularly efficacious pack configurations are specifically tailored so that when they are heated or cooled, the packs return to a temperature approximating either a room or body temperature in approximately 20 minutes (or, for example, between 17 and 30 minutes). Configured as such, actual timing by a user or patient of temperature treatments is not needed (but is still recommended for safety) because the hot or cold properties of the packs will dissipate at or by the conventional and/or doctor recommended time automatically (at which point, the treatment can be switched to the opposite temperature treatment, or repeated, or simply discontinued). That is, using such tailored configurations, the user or patient will receive the correct time application of the heat or cold temperature treatment but without the need to specifically time the treatment (which allows for more relaxation during treatment, for example).

In at least one such configuration, a pack is provided in which the beads fill the majority of the enclosed pack (e.g., 60-95%) with the remaining space being filled with water or gel (but with some room left within the pack, so that the pack is flexible and/or bendable, and/or deformable and the like). In certain embodiments, the liquid (e.g., water) or gel is used as a suspension. In certain embodiments using water, a bicarbonate buffer solution is added or employed. In at least one other example of such a configuration, the packs include beads filling approximately 80-99 percent of the interior pack area in combination with liquid or gel filling approximately 1-20 of the pack.

Example (Non-Limiting) Methods of Using the Packs

For Heat Therapy:

Wrap the pack with a damp cloth or paper towel. Microwave for 15 seconds. Since microwaves vary in power, you may microwave your pack for additional 5 second intervals until a desired temperature is reached. Adjust to comfort by placing a thin clean cloth or paper towel between the pack and the skin if necessary. It is recommended to apply the pack for 20 minutes (or as directed by your doctor), and then remove from the treatment area for 40-45 minutes (or as directed by your doctor). Packs can be reapplied after being warmed again.

For Cold Therapy:

Place your pack flat in freezer for at least 2 hours. Adjust to comfort by placing a thin clean cloth or paper towel between the pack and the skin if necessary. It is recommended to apply the pack for 20 minutes (or as directed by your doctor), and then remove it from the treatment area for 40-45 minutes (or as directed by your doctor). For better results, return the pack to the freezer after every use. The cold pack can be reapplied after being cooled again.

Pack Care:

When stored in the freezer, do not place any heavy objects on top of the pack. After use, the packs can be cleaned with a mild soap, detergent or alcohol.

Once given the above disclosure, many other features, modifications, and improvements will become apparent to the skilled artisan. Such features, modifications, and improvements are therefore considered to be part of this invention, without limitation imposed by the example embodiments described herein. Moreover, any word, term, phrase, feature, example, embodiment, or part or combination thereof, as used to describe or exemplify embodiments herein, unless unequivocally set forth as expressly uniquely defined or otherwise unequivocally set forth as limiting, is not intended to impart a narrowing scope to the invention in contravention of the ordinary meaning of the claim terms by which the scope of the patent property rights shall otherwise be determined:

I claim:

1. A hot and cold temperature treatment pack comprising:
a sealed pack shell comprised of one or more flexible walls configured so as to provide an interior compartment for carrying a heatable and chillable payload;
a plurality of deformable beads carried in said interior compartment;
a liquid carried in said interior compartment;
wherein said interior compartment is filled with a particular quantity of said deformable beads and a particular quantity of said liquid, and said ratio of deformable beads to said liquid within said interior compartment is selected such that:
a) when said treatment pack is heated, said treatment pack remains at a therapeutic, heated temperature for a therapeutically effective time; and
b) when said treatment pack is chilled, said treatment pack remains at a therapeutic, cooled temperature for a therapeutically effective time.

2. The treatment pack according to claim 1 further wherein said deformable beads fill at least approximately 60-95% of said interior compartment with the remaining percentage of said interior compartment being mostly filled with said liquid but with some percentage of said interior compartment remaining unfilled such that the treatment pack is conformable when applied to particular body parts.

3. The treatment pack according to claim 2 wherein said liquid is water or gel used in a quantity to form a suspension.

4. The treatment pack according to claim 3 wherein said treatment pack is configured and filled to a particular level to conform to a body part selected from the group consisting of: a facial area, an ankle, a knee, a shoulder, an elbow, and a head.

5. The treatment pack according to claim 4 wherein said deformable beads are hollow.

6. The treatment pack according to claim 4 wherein said deformable beads are non-hollow.

7. The treatment pack according to claim 5 wherein said deformable beads are comprised of glycerin.

8. The treatment pack according to claim 6 wherein said deformable beads are comprised of glycerin.

9. The treatment pack according to claim 4 wherein said interior compartment is filled 80-99 percent with said deformable beads and 1-20% with said liquid.

10. The treatment pack according to claim 9 wherein said one or more flexible walls are comprised of polyvinyl chloride.

11. The treatment pack according to claim 10 further including a percentage of bicarbonate buffer solution added to said liquid.

12. The treatment pack according to claim 2 wherein said ratio of deformable beads to said liquid within said interior compartment is selected such that the approximate time of heat or cold dissipation, after said treatment pack is heated or chilled, is approximately equal to a time equal to a doctor recommended temperature therapy.

13. The treatment pack according to claim 12 wherein said ratio of deformable beads to said liquid within said interior compartment is selected such that the approximate time of heat or cold dissipation is between, approximately, 17 and 30 minutes.

14. A hot and cold temperature treatment pack comprising:
a sealed pack shell comprised of one or more flexible walls configured so as to provide
an interior compartment for carrying a heatable and chillable payload;
a plurality of deformable beads carried in said interior compartment;
a liquid carried in said interior compartment;
wherein said interior compartment is filled with a particular quantity of said deformable beads and a particular quantity of said liquid, and said ratio of deformable beads to said liquid within said interior compartment is selected such that:
a) when said treatment pack is heated, said treatment pack remains at a therapeutic, heated temperature for a therapeutically effective time; and
b) when said treatment pack is chilled, said treatment pack remains at a therapeutic, cooled temperature for a therapeutically effective time; and
wherein said interior compartment is filled 80-99 percent with said deformable beads and. 1-20% with said liquid.

15. The treatment pack according to claim 14 wherein said ratio of deformable beads to said liquid within said interior compartment is selected such that the approximate time of heat or cold dissipation, after said treatment pack is heated or chilled, is between, approximately, 17 and 30 minutes.
